(19) United States
(12) Patent Application Publication
(10) Pub. No.: US 2018/0308564 A1
(43) Pub. Date: Oct. 25, 2018

(54) POINT-OF-CARE WORKSTATION/CART WITH SMARTPHONE INTERFACE

(71) Applicant: TOUCHPOINT MEDICAL, INC., Concordville, PA (US)

(72) Inventor: Graham Ross, Wikes-Barre, PA (US)

(73) Assignee: TOUCHPOINT MEDICAL, INC., Concordville, PA (US)

(21) Appl. No.: 15/767,774

(22) PCT Filed: Oct. 14, 2016
(86) PCT No.: PCT/US2016/057106

§ 371 Date: Apr. 12, 2018

Related U.S. Application Data
(60) Provisional application No. 62/242,375, filed on Oct. 16, 2015.

(51) Int. Cl. G16H 10/60; G06F 17/30; G06F 13/10; G16H 40/63; G07C 9/00

(52) U.S. Cl. CPC G16H 10/60; G06F 17/30876; H04M 1/72527; G16H 40/63; G07C 9/00912; G06F 13/102

(57) ABSTRACT

One example includes a point-of-care cart assembly configured for use by a medical professional. The point-of-care cart assembly including a mobile point-of-care cart configured to support at least one peripheral device facilitating the input of medical data specific to a patient, and an interface module mounted to the mobile point-of-care cart. When the mobile computing device is proximal to the mobile point-of-care cart, the interface module is configured to receive input from the medical professional including an access request for the mobile computing device to access medical data stored at a remote database server or a storage request for the mobile computing device to store new medical data in the remote database server, transmit the input to the mobile computing device for transfer to the remote database server, and receive the requested medical data from the remote database server or a confirmation that the new medical data is stored.

### CROSS REFERENCE TO RELATED APPLICATIONS

This application claims benefit of priority to U.S. Provisional Application No. 62/242,375, filed Oct. 16, 2015, the contents of such application being incorporated by reference herein.

### FIELD OF THE INVENTION

This invention relates generally to a point-of-care cart assembly configured for use by a medical professional in connection with delivering medical care to a patient, a workstation assembly configured to provide secure access to a controlled inventory of stored items, and a method for providing secure access to a controlled inventory of stored items. In particular aspects, this invention relates to systems and methods for providing a Point-Of-Care (POC) cart or workstation with an interface via a mobile computing device; specifically, the POC cart or workstation communicates with the mobile computing device to allow a user to control peripheral devices on the POC cart or workstation, and to allow access to database information through the POC cart or workstation.

### BACKGROUND OF THE INVENTION

It is beneficial for Point-Of-Care (POC) carts or workstations to include computer access such as for connecting to a hospital network. Accordingly, there remains a need for improved POC carts or workstations in terms of the security of access to the hospital network if the authorized user leaves the POC cart or workstation unattended.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 shows a POC cart/workstation communicating with a mobile device via an interface module to control an inventory bin module.

FIG. 2 shows the inventory bin module connected to the interface module which includes an interface board and a single board computer.

FIG. 3 shows a block diagram of the interface module including the interface board and the single board computer.

FIG. 4 shows flow diagram of communication between the mobile device, the interface module and the inventory bin module.

FIG. 5 shows block diagram of the mobile application running on the mobile device.

FIG. 6 shows a flowchart for controlling the bin module in FIG. 1.

FIG. 7 shows a POC cart/workstation communicating (wired or wirelessly) with a mobile device via the interface module in order to communicate with peripheral devices on the cart/workstation.

FIG. 8 shows a flowchart for controlling the POC cart/workstation in FIG. 7.

FIG. 9 shows a POC cart/workstation communicating (wired or wirelessly) with a mobile device via the interface module to control a medical device.

FIG. 10 shows a flowchart for controlling the medical device in FIG. 9.

### SUMMARY OF THE INVENTION

One example includes a point-of-care cart assembly configured for use by a medical professional in connection with delivering medical care to a patient. The point-of-care cart assembly including a mobile point-of-care cart configured to be positioned and repositioned for use by the medical professional. The mobile point-of-care cart also being configured to support at least one peripheral device facilitating the input of medical data specific to the patient for delivery to a remote database server. The mobile point-of-care care also including an interface module mounted to the mobile point-of-care cart. The interface module being configured for electronic communication with the at least one peripheral device. The interface module also being configured for electronic communication with a mobile computing device. When the mobile computing device is proximal to the mobile point-of-care cart, the interface module is configured to receive input from the medical professional via the at least one peripheral device, the input including an access request for the mobile computing device to access medical data stored at the remote database server or a storage request for the mobile computing device to store new medical data in the remote database server, transmit the input to the mobile computing device for transfer to the remote database server, and receive, from the mobile computing device, the requested medical data from the remote database server or a confirmation that the new medical data is stored in the remote database server.

One example includes a workstation assembly configured to provide secure access to a controlled inventory of stored items. The workstation including a bin module including a plurality of bins each configured to store one or more of the stored items. The bin module also including an electronic lock associated with each of the plurality of bins. The workstation also including an interface module coupled to the bin module and configured for electronic communication with at least one peripheral device facilitating the input of data specific to the stored items. The interface module also being configured for electronic communication with a mobile computing device. When the mobile computing device is proximal to the workstation, the interface module is configured to receive input from a user via the at least one peripheral device, the input including an access request to access the stored items in the bin module, transmit the input to the mobile computing device for transfer to a remote database server, receive, from the mobile computing device, instructions to unlock the stored items in the bin module, transmit at least one control signal to the bin module to unlock the electronic lock of a selected bin of the plurality of bins, and transmit inventory information associated with the selected bin to the mobile computing device for transfer to the remote database server.

One example includes a point-of-care cart assembly configured for use by a medical professional in connection with delivering medical care to a patient. The point-of-care cart assembly including a mobile point-of-care cart configured to be positioned and repositioned for use by the medical professional. The mobile point-of-care cart also being configured to support at least one peripheral device facilitating the input of medical data specific to the patient for delivery to a remote database server and at least one medical apparatus having an electronic sensor. The point-of-care cart assembly also including an interface module mounted to the mobile point-of-care cart. The interface module being configured for electronic communication with the at least one peripheral device and the medical apparatus, and the interface module also being configured for electronic communication with a mobile computing device. When the mobile computing device is proximal to the mobile point-of-care cart, the interface module is configured to receive input from a user via the at least one peripheral device, the input including a request to control the medical apparatus or to receive data from the medical apparatus, transmit the input to the mobile computing device for transfer to the remote database server, receive, from the mobile computing device, instructions to control the medical apparatus or to receive data from the medical apparatus, transmit at least one control signal to the medical apparatus or to receive data from the medical apparatus, receive data from the medical apparatus, and transmit the data to the mobile computing device for transfer to the remote database server.

One example includes a method for providing secure access to a controlled inventory of stored items of a workstation including a bin module having a plurality of bins each configured to store one or more of the stored items. The bin module also including an electronic lock associated with each of the plurality of bins. The workstation also including an interface module coupled for communication to the bin module and configured for electronic communication with at least one peripheral device facilitating the input of data specific to the stored items. The interface module also being configured for electronic communication with a mobile computing device. The method including when the mobile computing device is proximal to the workstation, receiving, by the interface module, input from a user via the at least one peripheral device, the input including an access request to access the stored items in the bin module, transmitting, by the interface module, the input to the mobile computing device for transfer to a remote database server, receiving, by the interface module, from the mobile computing device, instructions to unlock the stored items in the bin module, transmitting, by the interface module, at least one control signal to the bin module to unlock the electronic lock of a selected bin of the plurality of bins, and transmitting, by the interface module, inventory information associated with the selected bin to the mobile computing device for transfer to the remote database server.

### DETAILED DESCRIPTION OF EMBODIMENTS OF THE INVENTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

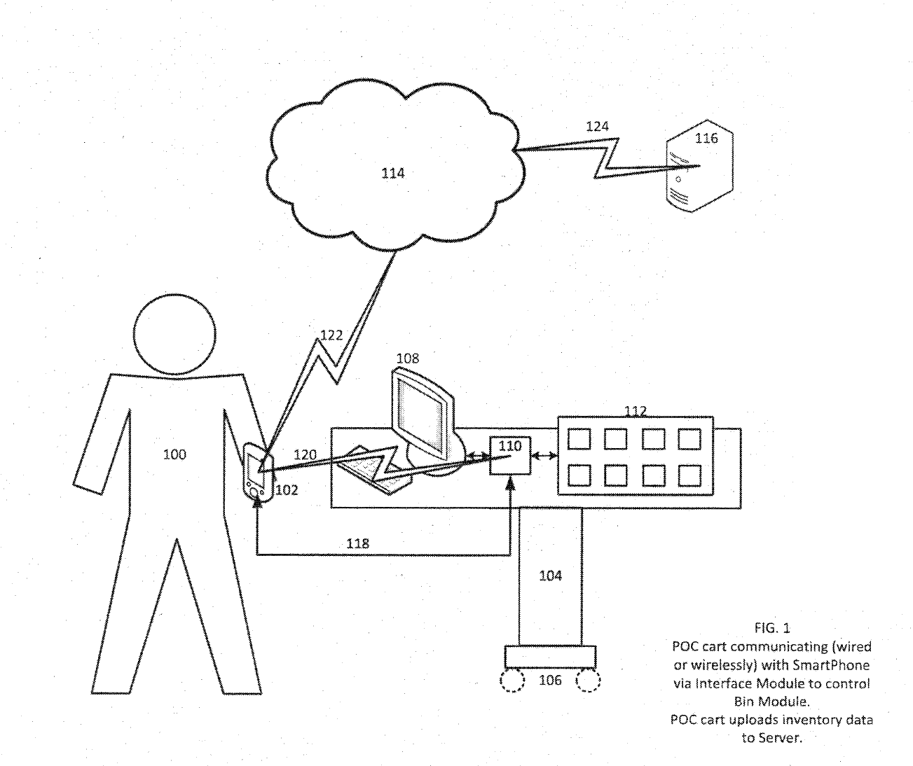

Shown in FIG. 1 is a Point-Of-Care (POC) cart/workstation 104 that may or may not include wheels 106. When the POC cart/workstation 104 includes wheels 106, it is considered a mobile cart that can be moved by the user. When the POC cart/workstation 104 does not include wheels 106, it is considered a static workstation that is not typically moved by the user.

POC cart/workstation 104 is typically for use by a user (e.g. nurse 100 or other caregiver) in a medical setting (e.g. hospital). In some embodiments, POC cart/workstation 104 includes peripheral devices such as keyboard/monitor 108, a mouse (not shown), and possibly other standard peripheral devices e.g., printer, barcode scanner, etc. (also not shown). POC cart/workstation 104 generally provides an interface for access to data on hospital network 114 and/or control over other peripheral devices (e.g. inventory bin module 112) through wired or wireless interfaces.

A user, such as nurse 100, is generally able to utilize the POC cart/workstation 104 to connect to remote database server 116 which includes database programs such as an Electronic Medication Administration Record (eMAR) system for accessing patient medical records. This information can be retrieved from database server 116 and displayed on the monitor 108 of POC cart/workstation 104. In addition to retrieving data from the database, nurse 100 can also input information (e.g. patient information) through the keyboard/mouse/scanner, and send this information to server 116 to update the database.

In the past, conventional POC carts/workstations were controlled by a cumbersome personal computer (PC) such as a laptop that a nurse would directly connected to the POC cart/workstation. When left unattended, however, these laptops posed a security risk allowing unauthorized individuals access to the hospital network and control over connected peripheral devices (e.g. inventory bin module 112).

In order to avoid this situation, in the present system, the laptop is replaced by a mobile computing device such as smartphone 102. As shown in FIG. 1, nurse 100 (as do most nurses) carries a mobile device (e.g. smartphone, personal digital assistant, etc.). Rather than carrying around a cumbersome laptop and connecting this laptop to the POC cart/workstation 104, a mobile device such as smartphone 102 is used to provide more seamless and convenient control over the POC cart/workstation 104.

For example, smartphone 102 may automatically connect wirelessly to the hospital network via wireless paths 122 and 124. This connection may be a WIFI, Bluetooth or other equivalent wireless connection. In some embodiments, smartphone 102 is plugged into a physical port on the interface module 110 of the POC cart/workstation 104 through a physical connection such as a micro-USB cable, Lightning cable, etc. The cable may convey one or more types of signals, such as digital video signals, USB signals, etc. between smartphone 102 and an interface module 110 which are then relayed to the keyboard/monitor 108 and other peripheral devices.

Generally, smartphone 102 is capable of sending video signals through the wired or wireless connection to an external display (e.g. the monitor). This may be the same image that is being displayed on the phone itself. This process is called mirroring, and is beneficial in that nurse 100 can view images from his/her smartphone 102 at POC cart/workstation 104 without having to remove smartphone 102 from his/her pocket.

In order to control the peripheral devices on POC cart/workstation 104, smartphone 102 communicates with interface module 110 that is mounted on POC cart/workstation 104. One or more peripheral devices such as the monitor/keyboard 108, mouse (not shown), scanner (not shown), printer (not shown), bioID reader (not shown) may also be plugged into (or wirelessly connected to) interface module 110. This enables standard wired or wireless peripheral devices to be used on the POC cart/workstation 104, thereby reducing cost and simplifying support.

For example, FIG. 1 shows physical cable 118 connecting smartphone 102 to interface module 110. Cable 118 may carry one or more types of signals. In one embodiment, cable 118 carries HDMI video signals that are routed by interface module 110 from smartphone 102 to monitor 108. In certain embodiments, cable 118 carries USB signals that are routed from one of more of the keyboard and mouse to smartphone 102. In certain embodiments, the interface module 110 may contain a USB hub to provide power to the peripheral devices.

In another example, FIG. 1 shows smartphone 102 connected to the interface module 110 via a wireless channel 120. In certain embodiments, the wireless channel is used to carry the signals that are routed via the interface module to monitor 108 and other peripheral devices of POC cart/workstation 104.

In addition to standard peripheral devices (e.g. monitor/keyboard 108, mouse, printer), POC cart/workstation 104 may also include non-standard peripheral devices in the form of medithal equipment (e.g. medical devices, inventory storage devices, etc.). An example of this is shown in FIG. 1 where POC cart/workstation 104 includes an inventory bin module 112 that includes multiple bins (e.g. drawers) for storing medical inventory (e.g. medication, needles, etc.). Each bin includes an electrically actuated lock (e.g. a solenoid that actuates a latch) for automatically locking and unlocking the bins, thereby allowing/preventing access to the inventory.

In an example of operation, smartphone 102 automatically connects to the interface module 110 via a wireless link when nurse 100 is close enough in proximity to POC cart/workstation 104 (e.g. within Bluetooth range). This may be a distance of 1-3 meters. Once smartphone 102 is connected to interface module 110, it sends a video image to the monitor and accepts input from the keyboard and any other peripheral devices attached to the connection module (e.g. the smartphone functionality is mirrored on the POC cart/workstation).

Interface module 110 could include firmware to proactively connect to mobile device through, for example, Bluetooth pairing. Alternately the mobile device could contain an application that connects via the wireless network to a remote web application, or database server 116 which then connects via wireless or wired connection to interface module 110. In this example, remote server 116 may interact with both interface module 110 and smartphone 102.

In addition to connecting to POC cart/workstation 104, smartphone 102 connects to server 116, accesses database information, and displays this database information (e.g. patient information) on monitor 108. This allows the nurse 100 to interact with the hospital network database by sending and receiving information to/from the server.

In order to provide this functionality, smartphone 102 executes a mobile application which provides control buttons for controlling the peripheral devices. For example, the mobile application may have the functionality that allows nurse 100 to communicate and control inventory bin module 112 to unlock or lock certain bins for access to medical inventory such as medication.

One benefit to this configuration, is increased security to both the network information and control over the peripheral devices. For example, when nurse 100 walks away from the POC cart/workstation 104, smartphone 102 eventually goes out of range and loses communication with the system. At this point, POC cart/workstation 104 cannot access the database or control the inventory bin module (e.g. the bins are automatically locked when communication to the phone is lost), because the POC cart/workstation 104 relies, on the processing power of smartphone 102 to perform these tasks. Thus, unlike conventional systems, the present system does not provide a risk of unauthorized access to the network or unauthorized control over the peripheral devices such as inventory bin module 112, because smartphone 102 is no longer able to control POC cart/workstation 104.

In some examples, nurse 100 may place smartphone 102 in a docking cradle (not shown) or connect smartphone 102 to the system through a cable. In this example, security is still maintained, because when nurse 100 unplugs smartphone 102 when leaving, POC cart/workstation 104 cannot access the database or control the inventory bin module. For example, the bins are automatically locked when the connection to the smartphone is lost, because the POC cart/workstation 104 relies on the processing of smartphone 102.

In some examples, smartphone 102 may store certain information in its internal memory. For example, smartphone 102 may store instructions that allow communication with and use of the monitor 108, keyboard, and other peripheral devices without simultaneous connection to the network. In some examples, smartphone 102 may store instructions that allow at least one function of the system to be used when smartphone 102 is not connected to the network. For example, smartphone 102 may allow nurse 100 to open an actuated bin of inventory bin module 112 to obtain medication for a patient and the then store a record of the access and medication retrieval until connection with the database is restored, whereupon the record is sent to the database server 116 via network 114.

As described above, the POC cart/workstation 104 may include a keyboard and monitor, thereby providing a large display and an input device for interaction with the programs running on the smartphone or interaction with programs running on the network, such as the eMAR. In order to make the system convenient, each room in a hospital may be equipped with a keyboard/monitor 108 and interface module 110, thereby enabling nurse 100 to have a large display and input device in each room without having to carry a computer or laptop with them from room to room. When the nurse 100 enters the room, smartphone 102 connects to the POC cart/workstation 104 in close proximity. When the nurse 100 leaves the room, the smartphone 102 goes out of communication range and disconnects from POC cart/workstation 104. The remaining monitor and keyboard on POC cart/workstation 104 do not present a risk of unauthorized access to the network, because they cannot access the network or control the peripheral devices without the aid of smartphone 102.

In certain examples, smartphone 102 does not store any sensitive data in the local memory. When smartphone 102 leaves the hospital and goes out of range of the hospital wireless network, there is no risk that sensitive information is carried with the smartphone 102. This makes the system compliant with requirements of the Health Insurance Portability and Accountability Act (HIPAA).

In certain examples, the software running on the smartphone 102 may require an initial login and authentication process when the user first enters the hospital and the smartphone 102 connects to the hospital network. In certain examples, this may be accomplished by prompting for a username and password. In other examples, this may be accomplished by connecting to a biometric peripheral device such as a fingerprint reader attached to the interface module (not shown). In other embodiments, the biometric reader may be integrated into smartphone 102 itself. In either scenario, the user is required to interact with the biometric reader or login procedure which transmits the input to an authentication service over the network 114. If the authentication service confirms that the user (e.g. the nurse) is authorized, the smartphone 102 is enabled for operation within the hospital to communicate with the network and control peripheral devices of the POC cart/workstation 104.

In certain examples, smartphone 102 remains in active communication with the network regardless of whether it is communicating with POC cart/workstation 104. For example, at the beginning of the work shift, nurse 100 could authenticate smartphone 102 to a remote authentication server (not shown). Interface module 110 can access the same remote authentication server via an application program interface (API) and receive information that informs the firmware of interface module 110 of the level of access granted to the authenticated user.

This eliminates the need for the nurse 100 to log-in each time they use anew POC cart/workstation 104 (i.e. nurse doesn't have to authenticate himself when walking from room to room). If multiple POC carts/workstations 104 are positioned around a facility such as a hospital, it allows nurse 100 to have the benefit of a large display and keyboard within a few steps of their location without having to carry around a laptop, tablet, or other bulky computer device or to push POC cart/workstation 104 with them. In certain examples, the nurse may interact directly through smartphone 102 to access the database, and then seamlessly transition to the larger display and keyboard interface by walking up to a nearby POC cart/workstation 104.

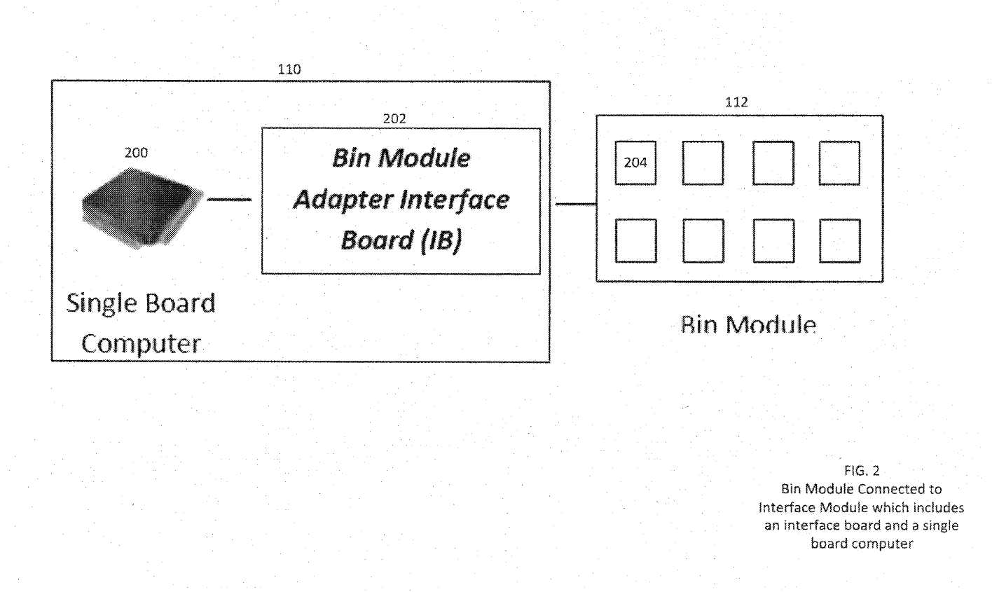

As described above, POC cart/workstation 104 may include both standard peripheral devices such as keyboard/monitor 108, and non-standard peripheral devices such as inventory bin module 112, which are all connected to interface module 110. Details of interface module 110 are shown in FIG. 2 to include a single board computer 200 for performing the processing and control over the peripheral devices, and an interface board 202 for interfacing single board computer 200 with the peripheral devices (e.g. bin module 112). Alternatively, interface board 202 of interface module 110 may be a functional section of single board computer 200 rather than a physically separate board.

Generally, single board computer 200 receives instructions from smartphone 102 and sends data (e.g. control signals) to the peripheral devices. For example, smartphone 102 may instruct single board computer 200 to open a specific bin on bin module 112 to dispense medication to a nurse. In response to this instruction, single board computer 200 may send a control signal to bin module 112 via interface board 202, instructing bin module 112 to unlock the specified bin 204.

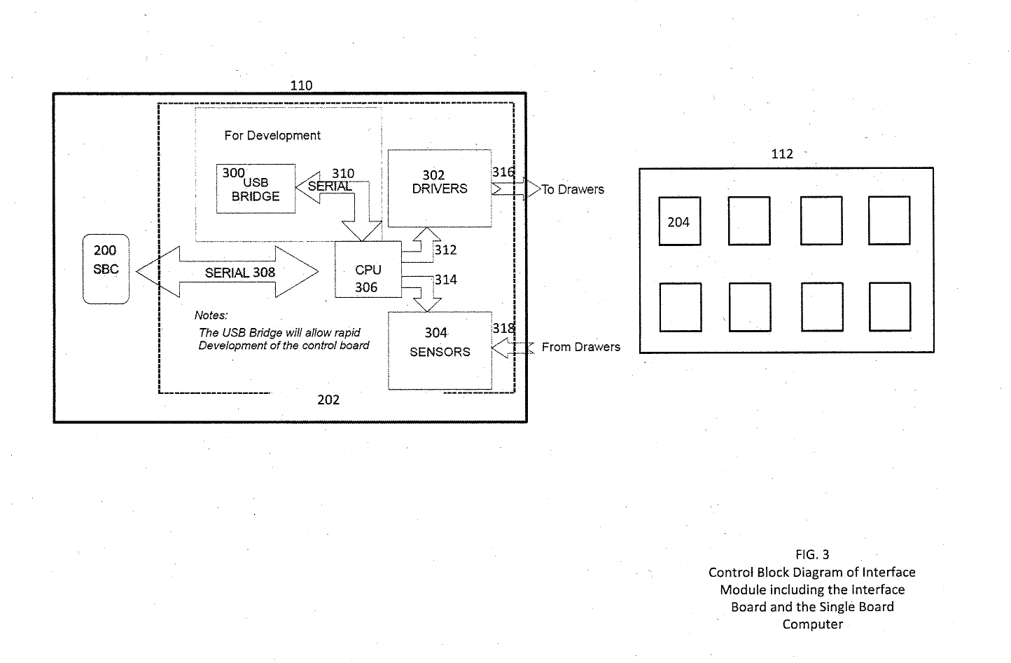

A more detailed illustration of interface module 110 is shown in FIG. 3. For example, interface board 202 may include a central processing unit (CPU) 306 that controls a universal serial bus (USB) bridge 300 via serial bus 310, controls bin module drivers 302 via data bus 312, controls bin module sensors 304 via data bus 314, and communicates with the single board computer 200 via serial data bus 308. Interface board 202 also includes port 316 for sending control signals to lock/unlock bins of the inventory bin module 112, and port 318 for receiving sensed information (i.e. open/closed status, presence/absence of inventory, etc.) for the bins of the inventory bin module 112. Although not shown, single board computer 200 includes components that provide communication with smartphone 102, processing of data, and control over peripheral devices. These components include but are not limited to a processor, input/output ports/interfaces, memory devices, and a wireless transceiver.

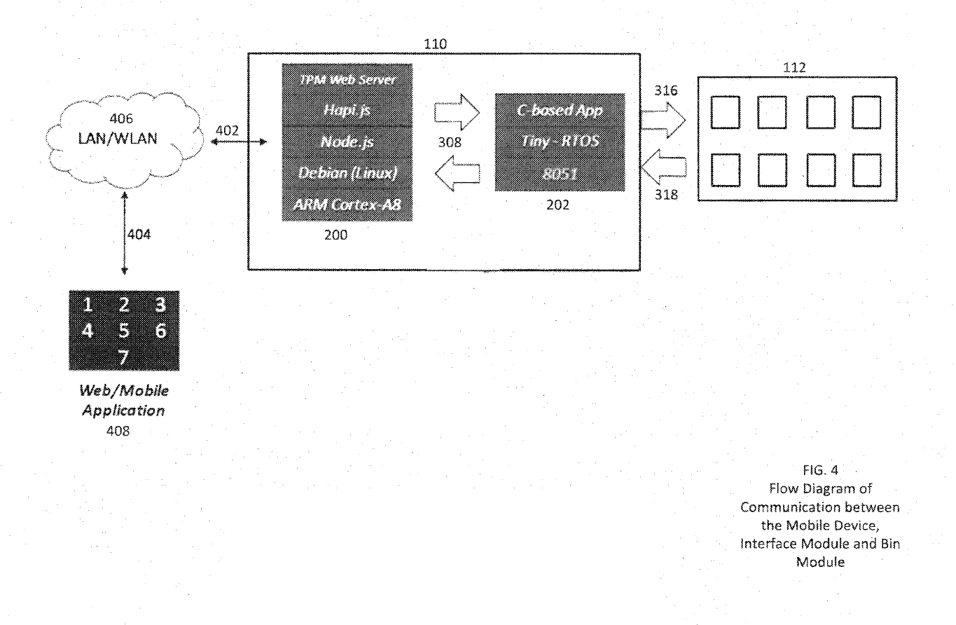

FIG. 4 shows another illustration of interface module 110 communicating with inventory bin module 112 and a mobile application 408 running on smartphone 102. In order to perform communication with smartphone 102, and control, inventory bin module 112, single board computer 200 and interface board 202 execute various software programs. For example, single board computer 200 includes a processor such as an ARM Cortex-A8. This processor may run an operating system such as Debian (Linux) that supports an event driven architecture such as Node.js for supporting real time web applications, a software for writing reusable application logic such as Hapi.js, and software for communicating with a web server. Similarly, interface board 202 includes a microcontroller such as Intel's 8051 that runs a real-time-operating-system (RTOS) and a C-language based application for controlling bin module 112.

During operation, mobile application 408 of smartphone 102 communicates with single board computer 200. This communication can be performed directly through a wired connection or wirelessly through Bluetooth. Alternatively, this communication can be performed through local area network (LAN) 406 via wired or wireless connections 402 and 404.

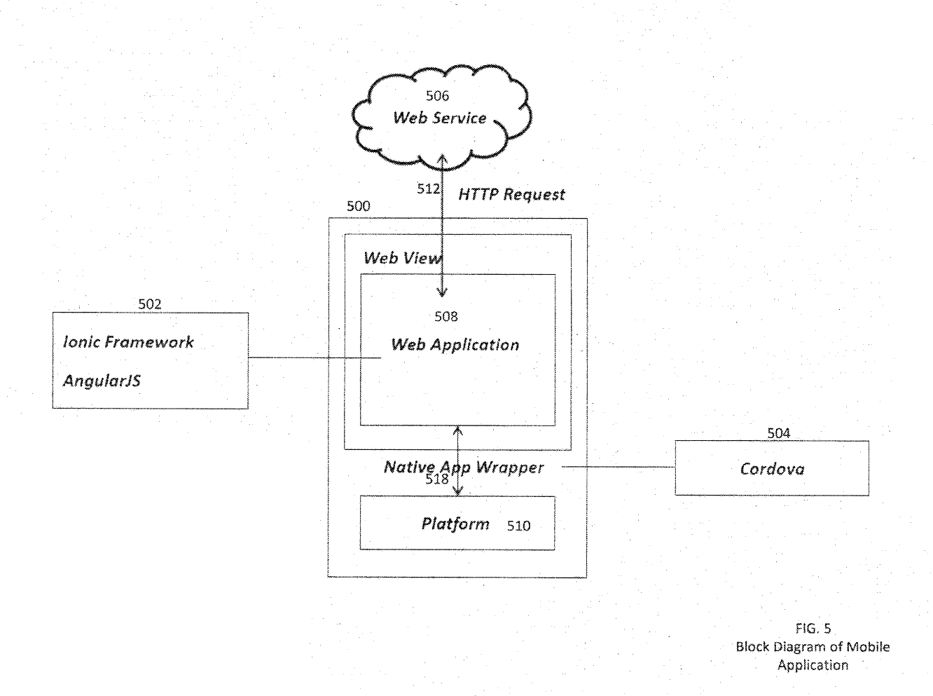

A more detailed explanation of the mobile application software running on smartphone 102 is shown in FIG. 5. For example, FIG. 5 shows that the software 408 includes a primary framework 500 that includes a web application 508 (i.e. a software application where the user interface runs in a web browser), native application wrapper 518, platform 510, Ionic framework 502 and Cordova framework 504. During operation, web application 508 runs on platform 510 and uses the native application wrapper 518 as well as the Ionic framework 502 and Cordova framework 504 to communicate with web service 506 via an HTTP request 512.

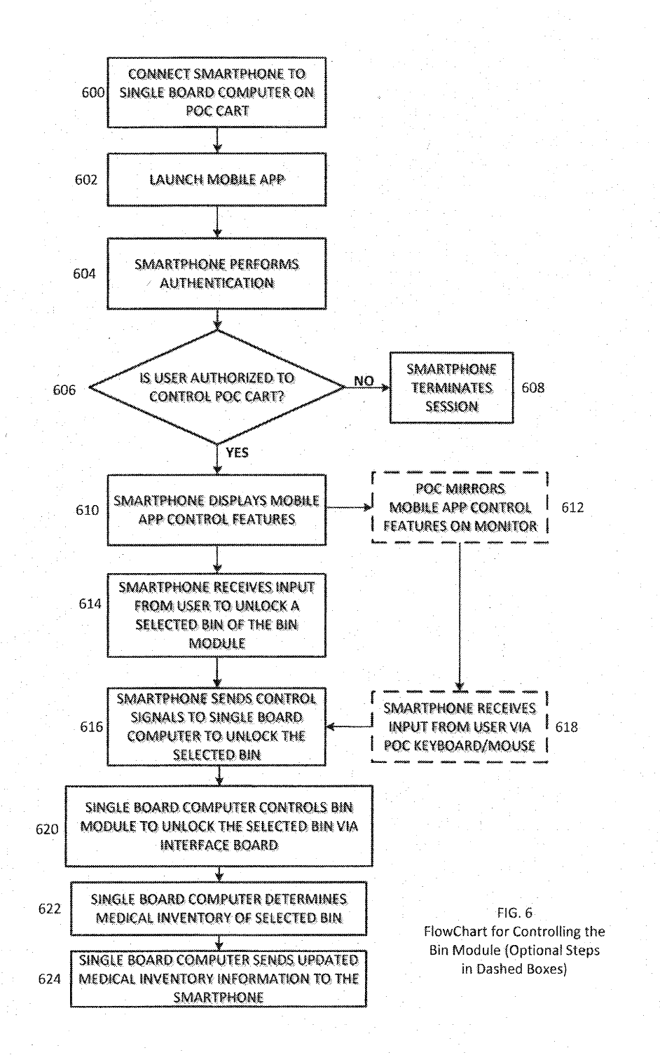

Prior to nurse 100 being able to access bin module 112 to retrieve medical inventory such as medication, smartphone 102 performs certain steps to authenticate itself and then communicate with interfaced module 110. These steps are illustrated in the flowchart of FIG. 6.

In step 600, when nurse 100 is in proximity to POC cart/workstation 104, smartphone 102 is connected (wired or wirelessly) to the single board computer of interface module 110. Upon connection, smartphone 102 launches a mobile application (step 602) that is configured to view bin inventory and control bin module 112. Once mobile application is launched, smartphone 102 performs authentication (step 604) of the nurse 100. In step 606, smartphone 102 determines if nurse 100 is authenticated. If nurse 100 is not authenticated, then smartphone 102 terminates the session in step 608. If, however, nurse 100 is authenticated, then smartphone 102 executes step 610 and displays the mobile application control features to control the bin module.

It should be noted that optionally (steps 612 and 618), smartphone 102 may mirror the display of the mobile application control features on the monitor of POC cart/workstation 104 and receive input from the keyboard of POC car/workstation 104. This, however, is not required. The nurse can control the bin module 112 directly via the display of smartphone 102.

In step 614, smartphone 102 receives input from nurse 100 requesting access to specific inventory stored in bin module 112. Smartphone 102 determines which bin the inventory is located in, and sends a control signal (step 616) to the single board computer of interface module 110. In step 620, the single board computer sends a control signal via the interface board to a selected one of the bins that contains the requested medical inventory. This control signal controls the electric lock of that bin to unlock, thereby providing access to the inventory (e.g. medication).

In order to keep track of inventory in the bin, the single board computer (step 622) detects (e.g. using sensors) the amount and type of inventory still remaining in the unlocked bin after the nurse gains access. In step 624, this updated medical inventory is sent to the smartphone 102 and then to server 116 in order to update the patient database.

For example, the bin inventory could be tracked using a sensor e.g., radio frequency identification (RFID) receiver attached to the bin, the cart work surface, or to each bin drawer. In this example, each inventory item would have an RFID tag attached.

In other examples, inventory (e.g. medication) stored in the bin module could be subject to business rules in the workflow that requires each user (e.g. each nurse) to perform pre- and/or post-counts of a bin drawer contents each time it is opened. These counts are manually entered (e.g. scanned, typed, etc.) into POC cart/workstation 104 and then transmitted to remote database 116. If a discrepancy occurs, an audit entry is automatically made and the bin can be locked from any additional access until the discrepancy is resolved by an authorized administrator. Users with sufficient rights may override the discrepancy lockout, and the override is recorded as an auditable transaction. Mobile alerts may also be generated and sent to administrators when such a discrepancy occurs.

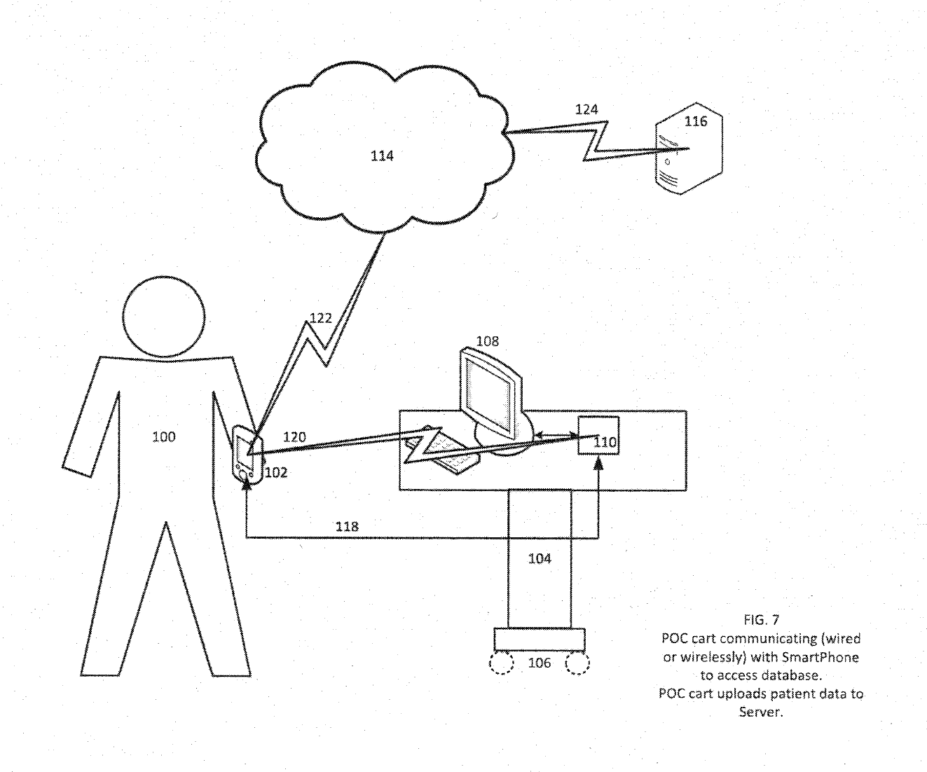

Although FIG. 1 shows a POC cart/workstation 104 having a medical inventory bin module 112, it is noted that the POC cart/workstation 104 does not require one. As shown in FIG. 7, the POC cart/workstation 104 may simply have a monitor/keyboard 108 and an interface board 110. This type of POC cart/workstation 104 allows the nurse 100 to access information from the database of server 116, and update database information.

For example, nurse 100 may require information about a certain patient. When nurse 100 walks up to POC cart/workstation 104, smartphone 102 automatically connects to interface module 110 and server 116. This allows smartphone 102 to mirror medical information on monitor 108, and allow the nurse to access and possible manipulate (e.g. update) this medical information using the keyboard on POC cart/workstation 104. In this example, nurse 100 does not need to take smartphone 102 out of his/her pocket.

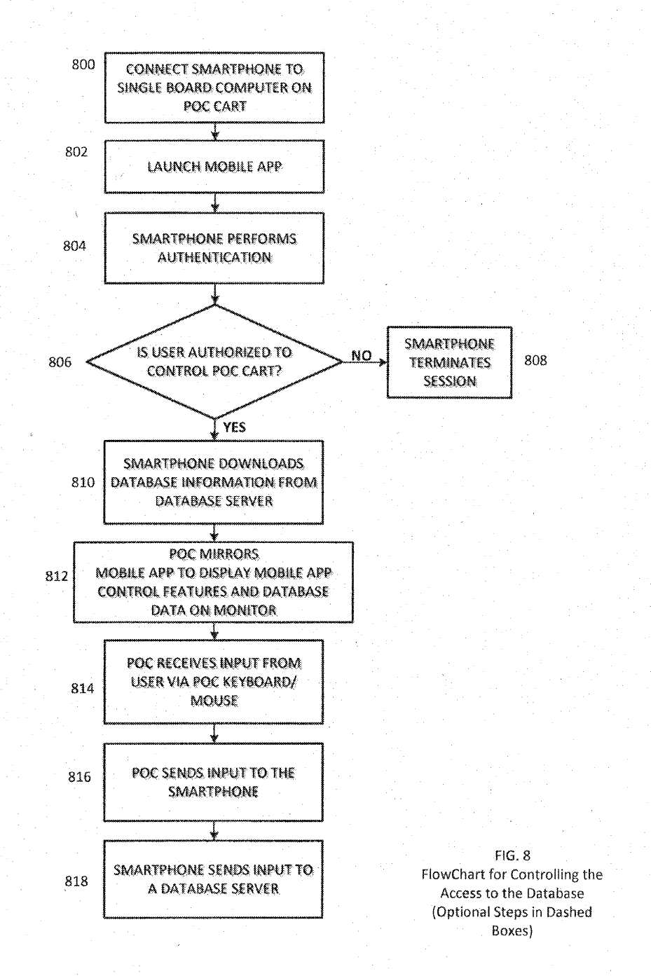

Prior to nurse 100 being able to access the database to retrieve or update medical information, the smartphone 102 performs certain steps to authenticate itself and then communicate with interface module 110. These steps are illustrated in the flowchart of FIG. 8.

In step 800, when nurse 100 is in proximity to POC cart/workstation 104, smartphone 102 is connected (wired or wirelessly) to the single board computer of interface module 110. Upon connection, smartphone 102 launches a mobile application (step 802) that is designed to view and access database information. Once the mobile application is launched, smartphone 102 performs authentication (step 804) of nurse 100. In step 806, smartphone 102 determines if nurse 100 is authenticated. If nurse 100 is not authenticated, then smartphone 102 terminates the session in step 808. If, however, nurse 100 is authenticated, then smartphone 102 executes step 810 and launches a mobile application to communicate with the database server 116.

In step 812, smartphone 102 mirrors the mobile application onto monitor 108 of POC cart/workstation 104. In step 814, nurse 100 is able to input information to the mobile application via the keyboard of POC cart/workstation 104. This information can be requests for database information or new information for updating the database. In step 816, POC cart/workstation 104 sends this input to smartphone 120, and in step 818 the smartphone 102 sends this input to database server 116. This process allows nurse 100 to access database information specific to a patient, and update the patients information.

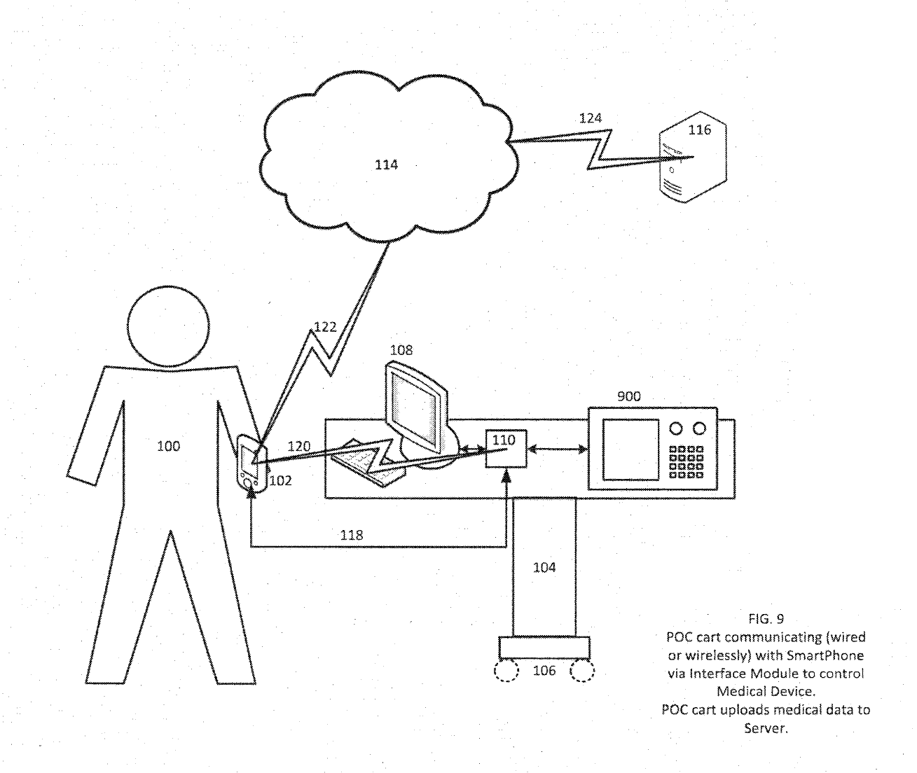

In another example, POC cart/workstation 104 can include other peripheral devices such as medical devices. As shown in FIG. 9, for example, POC cart/workstation 104, in addition to monitor/keyboard 108 and interface board 110, may include a medical device such as electrocardiogram (EKG) machine 900. This type of POC cart/workstation 104 allows nurse 100 to control and/or monitor the EKG machine from POC cart/workstation 104 and upload the EKG results to database server 116.

For example, nurse may wish to perform an EKG on a patient. When nurse 100 walks up to POC cart/workstation 104, smartphone 102 is connected to interface module 110 and server 116. This allows smartphone 102, for example, to mirror EKG controls on monitor 108, and allow the nurse to manipulate the EKG machine 900 using the keyboard on the POC cart/workstation 104. In this case, the nurse 100 does not need to take the smartphone 102 out of their pocket.

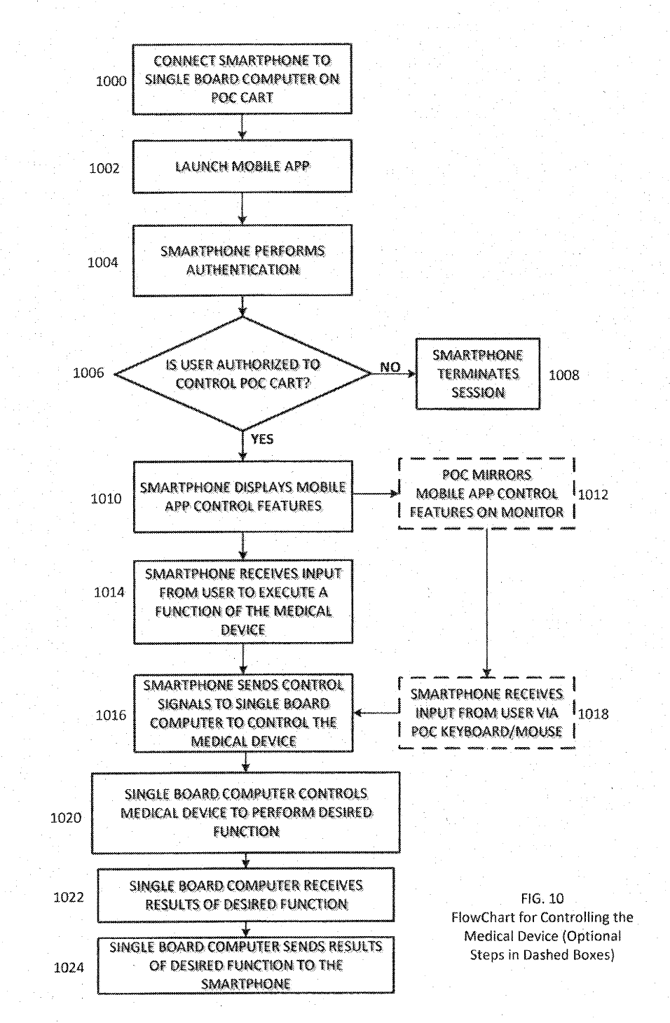

Prior to nurse 100 being able to control the EKG machine, smartphone 102 must perform certain steps to authenticate itself and then communicate with interface module 110. These steps are illustrated in the flowchart of FIG. 10.

In step 1000, when nurse 100 is in proximity to POC cart/workstation 104, smartphone 102 is connected (wired or wirelessly) to the single board computer of interface module 110. Upon connection, smartphone 102 launches a mobile application (step 1002) that is designed to control and/or monitor EKG machine 900. Once the mobile application is launched, smartphone 102 performs authentication (step 1004) of the nurse 100. In step 1006, smartphone 102 determines if nurse 100 is authenticated. If nurse 100 is not authenticated, then smartphone 102 terminates the session in step 608. If, however, nurse 100 is authenticated, then smartphone 102 executes step 1010 and displays the mobile application control features.

It should be noted that optionally (steps 1012 and 1018), smartphone 102 may mirror the display of the mobile application control features on the monitor of POC cart/workstation 104 and receive input from the keyboard of POC car/workstation 104. This, however, is not required. The nurse can control the EKG machine 900 directly by smartphone 102.

In step 1014, smartphone 102 receives input from nurse 100 requesting to execute a function of EKG machine 900. Smartphone 102 sends a control signal (step 1016) to the single board computer of interface module 110 to control EKG machine 900. In step 1020, the single board computer sends a control signal via the interface board to EKG machine 900. This control signal controls EKG machine 900 to perform the desired function (e.g. monitor the patient's heart).

Once EKG machine 900 completes the scan, the single board computer (step 1022) receives the results of the EKG. In step 1024, the EKG results are sent by the single board computer to the smartphone 102, and eventually to the server 116 in order to update the patient database with the results.

Referring generally to the figures, and in order to provide an overview of exemplary features of this invention, the following paragraphs summarize selected aspects of the invention.

One example includes a point-of-care cart assembly configured for use by a medical professional 100 in connection with delivering medical care to a patient. The point-of-care cart assembly including a mobile point-of-care cart 104 configured to be positioned and repositioned for use by the medical professional 100. The mobile point-of-care cart 104 also being configured to support at least one peripheral device 108 facilitating the input of medical data specific to the patient for delivery to a remote database server 116. The mobile point-of-care care also including an interface module 110 mounted to the mobile point-of-care cart 104. The interface module 110 being configured for electronic communication with the at least one peripheral device 108. The interface module 110 also being configured for electronic communication with a mobile computing device 102. When the mobile computing device 102 is proximal to the mobile point-of-care cart 104, the interface module 110 is configured to receive input from the medical professional 100 via the at least one peripheral device 108, the input including an access request for the mobile computing device 102 to access medical data stored at the remote database server 116 or a storage request for the mobile computing device 102 to store new medical data in the remote database server 116, transmit the input to the mobile computing device 102 for transfer to the remote database server 116, and receive, from the mobile computing device 102, the requested medical data from the remote database server 116 or a confirmation that the new medical data is stored in the remote database server 116.

In one example, the at least one peripheral device 108 is selected from the group consisting of a keyboard, a monitor, a microphone, a speaker, and a mouse. In one example, the interface module 110 is further configured to mirror, on the at least one peripheral device 108, as part of the facilitated electronic communication, functionality of a mobile application executed on the mobile computing device 102. In one example, the interface module 110 includes a processor 200 for processing the input and medical data, and an interface board 202 having at least one of data ports for plugging in the at least one peripheral device 108 or a wireless transceiver for communicating with the at least one peripheral device 108. In one example, when the mobile computing device 102 is not proximal to the mobile point-of-care cart 104, the interface module 110 is further configured to stop electronic communication between the mobile computing device 102 and the at least one peripheral device 108, and prevent access to the medical data stored in the remote database server 116 via the mobile point-of-care cart 104.

Another example includes a workstation assembly configured to provide secure access to a controlled inventory of stored items. The workstation including a bin module 112 including a plurality of bins each configured to store one or more of the stored items. The bin module 112 also including an electronic lock associated with each of the plurality of bins. The workstation also including an interface module 110 coupled to the bin module 112 and configured for electronic communication with at least one peripheral device 108 facilitating the input of data specific to the stored items. The interface module 110 also being configured for electronic communication with a mobile computing device 102. When the mobile computing device 102 is proximal to the workstation, the interface module 110 is configured to receive input from a user via the at least one peripheral device 108, the input including an access request to access the stored items in the bin module 112, transmit the input to the mobile computing device 102 for transfer to a remote database server 116, receive, from the mobile computing device 102, instructions to unlock the stored items in the bin module 112, transmit at least one control signal to the bin module 112 to unlock the electronic lock of a selected bin of the plurality of bins, and transmit inventory information associated with the selected bin to the mobile computing device 102 for transfer to the remote database server 116.

In one example, the at least one peripheral device 108 is selected from the group consisting of a keyboard, a monitor, a microphone, a speaker, a biometric sensor, and a mouse. In one example, the workstation is mounted to a mobile point-of-care cart 104 configured to be positioned and repositioned for use by a medical professional 100, and the stored items include at least one of medication or medical supplies. In one example, the interface module 110 includes a processor 200 for processing the input and instructions, and an interface board 202 having at least one of data ports for plugging in the at least one peripheral device or a wireless transceiver for communicating with the peripheral devices 108. In one example, when the mobile computing device 102 is not proximal to the mobile point-of-care cart 104, interface module 110 is further configured to stop electronic communication between the mobile computing device 102 and the at least one peripheral device 108, and prevent access to the stored items in the bin module 112 via the workstation assembly.

Yet another example includes a point-of-care cart assembly configured for use by a medical professional 100 in connection with delivering medical care to a patient. The point-of-care cart assembly including a mobile point-of-care cart 104 configured to be positioned and repositioned for use by the medical professional 100. The mobile point-of-care cart 104 also being configured to support at least one peripheral device 108 facilitating the input of medical data specific to the patient for delivery to a remote database server 116 and at least one medical apparatus 900 having an electronic sensor. The point-of-care cart assembly also including an interface module 110 mounted to the mobile point-of-care cart 104. The interface module 110 being configured for electronic communication with the at least one peripheral device 108 and the medical apparatus 900, and the interface module 110 also being configured for electronic communication with a mobile computing device 102. When the mobile computing device 102 is proximal to the mobile point-of-care cart 104, the interface module 110 is configured to receive input from a user via the at least one peripheral device 108, the input including a request to control the medical apparatus 900 or to receive data from the medical apparatus 900, transmit the input to the mobile computing device 102 for transfer to the remote database server 116, receive, from the mobile computing device 102, instructions to control the medical apparatus 900 or to receive data from the medical apparatus 900, transmit at least one control signal to the medical apparatus 900 or to receive data from the medical apparatus 900, receive data from the medical apparatus 900, and transmit the data to the mobile computing device 102 for transfer to the remote database server 116.

In one example, the at least one peripheral device 108 is, selected from the group consisting of a keyboard, a monitor, a microphone, a speaker, a biometric sensor, and a mouse. In one example, the interface module 110 is configured to control the medical apparatus 900 to perform at least one of an EKG or a Blood Pressure Analysis. In one example, the interface module 110 includes a processor 200 for processing the input, the instructions, and the data from the medical apparatus 900, and an interface board 202 having at least one of data ports for plugging in the at least one peripheral device 108 or a wireless transceiver for communicating with the at least one peripheral device. In one example, when the mobile computing device 102 is not proximal to the mobile point-of-care cart 104, interface module 110 is further configured to stop electronic communication between the mobile computing device 102 and the peripheral device 108, and prevent access to the stored items in the bin module 112 via the workstation assembly.

Still another example includes a method for providing secure access to a controlled inventory of stored items of a workstation including a bin module 112 having a plurality of bins each configured to store one or more of the stored items. The bin module also including an electronic lock associated with each of the plurality of bins. The workstation also including an interface module 110 coupled for communication to the bin module and configured for electronic communication with at least one peripheral device 108 facilitating the input of data specific to the stored items. The interface module 110 also being configured for electronic communication with a mobile computing device 102. The method including when the mobile computing device 102 is proximal to the workstation, receiving, by the interface module 110, input from a user via the at least one peripheral device, the input including an access request to access the stored items in the bin module 112, transmitting, by the interface module 110, the input to the mobile computing device 102 for transfer to a remote database server 116, receiving, by the interface module 110, from the mobile computing device 102, instructions to unlock the stored items in the bin module 112, transmitting, by the interface module 110, at least one control signal to the bin module 112 to unlock the electronic lock of a selected bin of the plurality of bins, and transmitting, by the interface module 110, inventory information associated with the selected bin to the mobile computing device 102 for transfer to the remote database server 116.

In one example, the method includes facilitating input and output of data to the workstation through at least one peripheral device 108 selected from the group consisting of a keyboard, a monitor, a microphone, a speaker, a biometric sensor, and a mouse. In one example, the method includes unlocking, by the interface module 110, the electronic lock of the selected bin of the plurality of bins, and allowing a medical professional 100 to access at least one of medication or medical supplies stored in the bin. In one example, the method includes processing, by a processor 200 of the interface module 110, the input and instructions, and providing, by an interface board, communication to the peripheral devices, the interface board 202 including at least one of data ports for plugging in the peripheral devices 108 or a wireless transceiver for wireless communication. In one example, when the mobile computing device 102 is not proximal to the mobile point-of-care cart 104, stopping, by the interface module 110, electronic communication between the mobile computing device 102 and the peripheral device 108, and preventing, by the interface module 110, access to the stored items in the bin module via the workstation assembly.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A point-of-care cart assembly configured for use by a medical professional in connection with delivering medical care to a patient, the point-of-care cart assembly comprising:
a mobile point-of-care cart configured to be positioned and repositioned for use by the medical professional, the mobile point-of-care cart also being configured to support at least one peripheral device facilitating the input of medical data specific to the patient for delivery to a remote database server; and
an interface module mounted to the mobile point-of-care cart, the interface module being configured for electronic communication with the at least one peripheral device, the interface module also being configured for electronic communication with a mobile computing device;
wherein, when the mobile computing device is proximal to the mobile point-of-care cart, the interface module is configured to:
receive input from the medical professional via the at least one peripheral device, the input including an access request for the mobile computing device to access medical data stored at the remote database server or a storage request for the mobile computing device to store new medical data in the remote database server,
transmit the input to the mobile computing device for transfer to the remote database server, and
receive, from the mobile computing device, the requested medical data from the remote database server or a confirmation that the new medical data is stored in the remote database server.

2. The point-of-care cart assembly of claim 1, wherein
the at least one peripheral device is selected from the group consisting of a keyboard, a monitor, a microphone, a speaker, and a mouse.

3. The point-of-care cart assembly of claim 1, wherein the interface module is further configured to:
mirror, on the at least one peripheral device, as part of the facilitated electronic communication, functionality of a mobile application executed on the mobile computing device.

4. The point-of-care cart assembly of claim 1, wherein the interface module includes:
a processor for processing the input and medical data; and
an interface board having at least one of data ports for plugging in the at least one peripheral device or a wireless transceiver for communicating with the at least one peripheral device.

5. The point-of-care cart assembly of claim 1, wherein when the mobile computing device is not proximal to the mobile point-of-care cart, the interface module is further configured to:
stop electronic communication between the mobile computing device and the at least one peripheral device; and
prevent access to the medical data stored in the remote database server via the mobile point-of-care cart.

6. A workstation assembly configured to provide secure access to a controlled inventory of stored items, the workstation comprising:
a bin module including a plurality of bins each configured to store one or more of the stored items, the bin module also including an electronic lock associated with each of the plurality of bins; and
an interface module coupled to the bin module and configured for electronic communication with at least one peripheral device facilitating the input of data specific to the stored items, the interface module also being configured for electronic communication with a mobile computing device;
wherein, when the mobile computing device is proximal to the workstation, the interface module is configured to:
receive input from a user via the at least one peripheral device, the input including an access request to access the stored items in the bin module,
transmit the input to the mobile computing device for transfer to a remote database server,
receive, from the mobile computing device, instructions to unlock the stored items in the bin module,
transmit at least one control signal to the bin module to unlock the electronic lock of a selected bin of the plurality of bins, and
transmit inventory information associated with the selected bin to the mobile computing device for transfer to the remote database server.

7. The workstation assembly of claim 6, wherein
the at least one peripheral device is selected from the group consisting of a keyboard, a monitor, a microphone, a speaker, a biometric sensor, and a mouse.

8. The workstation assembly of claim 6, wherein
the workstation is mounted to a mobile point-of-care cart configured to be positioned and repositioned for use by a medical professional, and the stored items include at least one of medication or medical supplies.

9. The workstation assembly of claim 6, wherein the interface module includes:
a processor for processing the input and instructions; and
an interface board having at least one of data ports for plugging in the at least one peripheral device or a wireless transceiver for communicating with the peripheral devices.

10. The workstation assembly of claim 6, wherein when the mobile computing device is not proximal to the mobile point-of-care cart, interface module is further configured to:
stop electronic communication between the mobile computing device and the at least one peripheral device; and
prevent access to the stored items in the bin module via the workstation assembly.

11. A point-of-care cart assembly configured for use by a medical professional in connection with delivering medical care to a patient, the point-of-care cart assembly comprising:
a mobile point-of-care cart configured to be positioned and repositioned for use by the medical professional, the mobile point-of-care cart also being configured to support at least one peripheral device facilitating the input of medical data specific to the patient for delivery to a remote database server and at least one medical apparatus having an electronic sensor; and
an interface module mounted to the mobile point-of-care cart, the interface module being configured for electronic communication with the at least one peripheral device and the medical apparatus, the interface module also being configured for electronic communication with a mobile computing device;
wherein, when the mobile computing device is proximal to the mobile point-of-care cart, the interface module is configured to:
receive input from a user via the at least one peripheral device, the input including a request to control the medical apparatus or to receive data from the medical apparatus,
transmit the input to the mobile computing device for transfer to the remote database server,
receive, from the mobile computing device, instructions to control the medical apparatus or to receive data from the medical apparatus,
transmit at least one control signal to the medical apparatus or to receive data from the medical apparatus,
receive data from the medical apparatus, and
transmit the data to the mobile computing device for transfer to the remote database server.

12. The point-of-care cart assembly of claim 11, wherein
the at least one peripheral device is selected from the group consisting of a keyboard, a monitor, a microphone, a speaker, a biometric sensor, and a mouse.

13. The point-of-care cart assembly of claim 11, wherein the interface module is configured to:
control the medical apparatus to perform at least one of an EKG or a Blood Pressure Analysis.

14. The point-of-care cart assembly of claim 11, wherein the interface module includes:
a processor for processing the input, the instructions, and the data from the medical apparatus; and
an interface board having at least one of data ports for plugging in the at least one peripheral device or a wireless transceiver for communicating with the at least one peripheral device.

15. The point-of-care cart assembly of claim 11, wherein when the mobile computing device is not proximal to the mobile point-of-care cart, interface module is further configured to:
stop electronic communication between the mobile computing device and the peripheral device; and
prevent access to the stored items in the bin module via the workstation assembly.

16. A method for providing secure access to a controlled inventory of stored items of a workstation including a bin module having a plurality of bins each configured to store one or more of the stored items, the bin module also including an electronic lock associated with each of the plurality of bins, the workstation also including an interface module coupled for communication to the bin module and configured for electronic communication with at least one peripheral device facilitating the input of data specific to the stored items, the interface module also being configured for electronic communication with a mobile computing device, the method including:
when the mobile computing device is proximal to the workstation:
receiving, by the interface module, input from a user via the at least one peripheral device, the input including an access request to access the stored items in the bin module,
transmitting, by the interface module, the input to the mobile computing device for transfer to a remote database server,
receiving, by the interface module, from the mobile computing device, instructions to unlock the stored items in the bin module,
transmitting, by the interface module, at least one control signal to the bin module to unlock the electronic lock of a selected bin of the plurality of bins, and
transmitting, by the interface module, inventory information associated with the selected bin to the mobile computing device for transfer to the remote database server.

17. The method of claim 16, including
facilitating input and output of data to the workstation through at least one peripheral device selected from the group consisting of a keyboard, a monitor, a microphone, a speaker, a biometric sensor, and a mouse.

18. The method of claim 16, including
unlocking, by the interface module, the electronic lock of the selected bin of the plurality of bins, and allowing a medical professional to access at least one of medication or medical supplies stored in the bin.

19. The method of claim 16, including
processing, by a processor of the interface module, the input and instructions; and
providing, by an interface board, communication to the peripheral devices, the interface board including at least one of data ports for plugging in the peripheral devices or a wireless transceiver for wireless communication.

20. The method of claim 16, including
when the mobile computing device is not proximal to the mobile point-of-care cart:
stopping, by the interface module, electronic communication between the mobile computing device and the peripheral device; and
preventing, by the interface module, access to the stored items in the bin module via the workstation assembly.
